(19) United States
(12) Patent Application Publication
(10) Pub. No.: US 2019/0091356 A1
(43) Pub. Date: Mar. 28, 2019

(54) ARRANGEMENT FOR DISINFECTING CAN LIDS FOR CLOSING CANS

(71) Applicants: KHS GMBH, DORTMUND (DE); CLARANOR SA, AVIGNON-CEDEX 9 (FR)

(72) Inventors: CHRISTOPHE RIEDEL, AVIGNON (FR); LUDWIG CLUESSERATH, BAD KREUZNACH (DE)

(21) Appl. No.: 16/085,667

(22) PCT Filed: Jan. 25, 2017
(86) PCT No.: PCT/EP2017/051459

§ 371 Date: Sep. 17, 2018

(30) Foreign Application Priority Data
Mar. 16, 2016 (DE) 10 2016 104 859.3

(51) Int. Cl. A61L 2/10; A61L 2/26; B65B 55/08; B67B 3/00

(52) U.S. Cl. CPC A61L 2/10; A61L 2/26; A61L 2202/23; B67B 3/003; A61L 2202/11; B65B 55/08

(57) ABSTRACT

An arrangement for disinfecting can lids and for closing cans includes a feed device which feeds a can closing unit. Receiving portions for individual can lids are arranged in the feed device. The feed device is situated in the feed region of the can closing unit. At least one sterilization device, for can lids received individually in the feed device, is arranged in a conveying region of the feed device.

The present invention relates to an arrangement for disinfecting can lids and for closing cans. Separate devices arranged at different positions of a filling plant for the containers have hitherto been provided to perform this task. A problem with these known arrangements is that the sterilized can lids have to be kept sterile until they are fed to the can closing unit. Their path must therefore be enclosed and if necessary a corresponding sterile gas supply must be provided in the transport region to prevent bacteria from reaching the can lids through the surrounding air.

It is therefore the object of the invention to create an arrangement in which the sterility of the can lids before they are closed is more effectively ensured.

This object is inventively resolved by an arrangement according to claim 1. Advantageous further embodiments of the invention are the subject-matter of the dependent claims.

According to the invention, the arrangement comprises a feed device, e.g. a transport star, in which receiving portions, e.g. transport recesses, are arranged for individual can lids. The feed device is arranged in the feed region of a can closing unit. According to the invention the arrangement comprises at least one sterilization device for the can lids received individually in the feed device, the sterilization device being arranged in the conveyor region of the feed device. The advantage of the invention is that the sterilization device is arranged directly in connection with the feed device for the can closing unit. As a result, the sterilized can lids move over only a minimal conveyor path before being fed, in particular from the feed device, to the can closing unit.

The sterilization device preferably contains a sterilizer, e.g. UV emitter, for the top and for the underside of the lid respectively. In this way the can lid is sterilized on both sides, although as a minimum requirement it would be sufficient for the can lid to be sterilized only on its underside facing the can because this affects the hygiene of the product filled into the can, for example a beverage. The sterilization device can of course also work with gas, chemicals and/or heat. The use of a UV emitter least affects the overall technical design of the inventive arrangement.

The feed device is preferably configured as a transport star which in its rotary motion is controlled in particular by a motor. A transport star is technically simple to realise and ensures a safe pick-up and feeding of the can lids to the can closing unit, and the transport star also comprises enough space on its periphery for the arrangement of the at least one sterilization device.

The can closing unit is preferably arranged in the conveyor region of the transport star so that the sterilized can lids are transferred directly from the feed star to the closing position of the can closing unit.

The conveyor path of the sterilized can lid is therefore reduced to a certain sector of the transport star. This facilitates a high sterility of the can lid when it is closed.

On its peripheral section the feed star preferably comprises transport recesses for the individual can lids, said recesses forming the receiving portions for the can lids. An arrangement of this kind is easy to realise and ensures a more secure holding of the can lids during their sterilization and, if applicable, when they are applied to the can in the can closing unit. Can lids can also be easily transferred from a stacker device to the transport star or from the transport star to a stacker device.

The sterilization device preferably contains at least one pulsed light device which contactlessly irradiates the lids using powerful light pulses, preferably in the UV range, at the areas to be sterilized and thus ensures sterility. The advantage of such a sterilization device is also that they in no way impede the progress and transport of the individual can lids in the transport star.

A first stacker device for can lids is preferably arranged in the entry of the feed device so that the can lids can be transferred individually from the first stacker device to the transport star.

In an advantageous embodiment of the invention a second stacker device which lies within the feed of the can closing unit is arranged at the exit from the feed device, e.g. the transport stars. In this case the second stacker device preferably forms the feed to the can closing unit. This also permits a very direct feed of the sterilized can lids to the can closing unit, the advantage of this arrangement being that the second stacker device arranged between the feed device and the can closing unit forms a buffer so that short-term variations in the sterilization and/or operating speed of the can closing units can be balanced out, limited of course by the stacking capacity of the second stacker device.

The can closing unit is preferably arranged directly in or at the exit from the feed device so that the can lids are not required to traverse long conveying paths once they have been discharged from the feed device.

In an advantageous embodiment of the invention the can closing unit is arranged at the feed device and the feed device feeds the sterilized can lids individually to the can closing unit. In this case the feed device already acts as a descrambling device for the feed to the can closing unit. Thus the feed device fulfils two tasks at the same time, namely the separating out and sterilizing of the can lids, and feeding them into the can closing unit.

The feed device and the sterilization device are preferably arranged in a sealed housing which comprises a supply and a discharge for a sterile gas and of course a feed opening and a discharge opening for the can lids. In this way the entire process can be carried out in a sterile space, thereby increasing the sterility of the can lids to be fitted in the can closing unit.

In an advantageous embodiment of the invention the first stacker device is arranged in the feed opening and/or the second stacker device is arranged in the discharge opening. This creates a lot of space in the housing and allows a simple feeding and removing of the can lids.

The openings which are provided for the feeding or removing of the can lids are preferably also used for gas feeding and removing. In this way the number of openings in the otherwise sealed housing can be minimised, thereby enhancing the maintenance of sterility. However it is not absolutely necessary for the feed opening for the can lids to be used as the feed opening for the gas as well.

The following terms are used synonymously: gas feed—gas feed channel; can lids—lids; outlet pipe—gas discharge—can lid discharge opening; sterilization device—sterilizer—UV emitter

It will be apparent to the skilled person that the above embodiments of the invention can be combined with one another in any desired way.

The invention is now described by reference to an exemplary embodiment in conjunction with the schematic drawings, in which

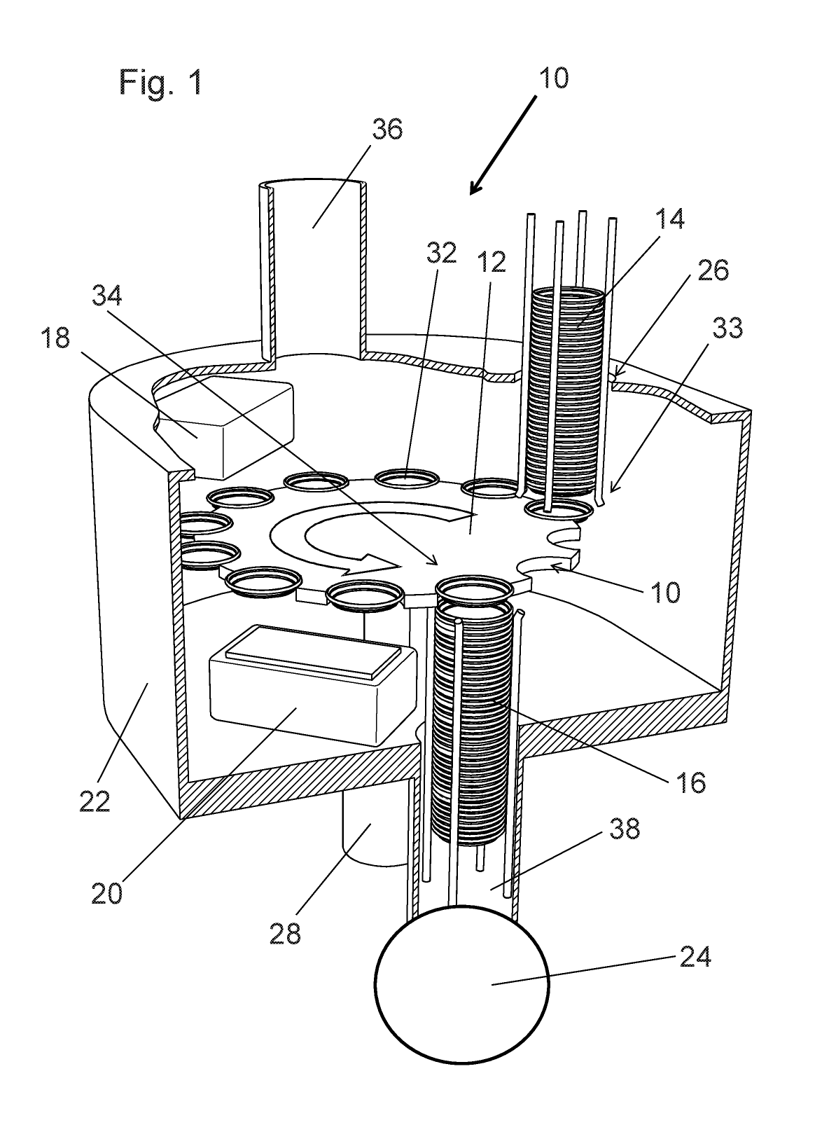

FIG. 1 shows a perspective view of an inventive arrangement having a stacker device arranged between the feed device and the can closing unit;

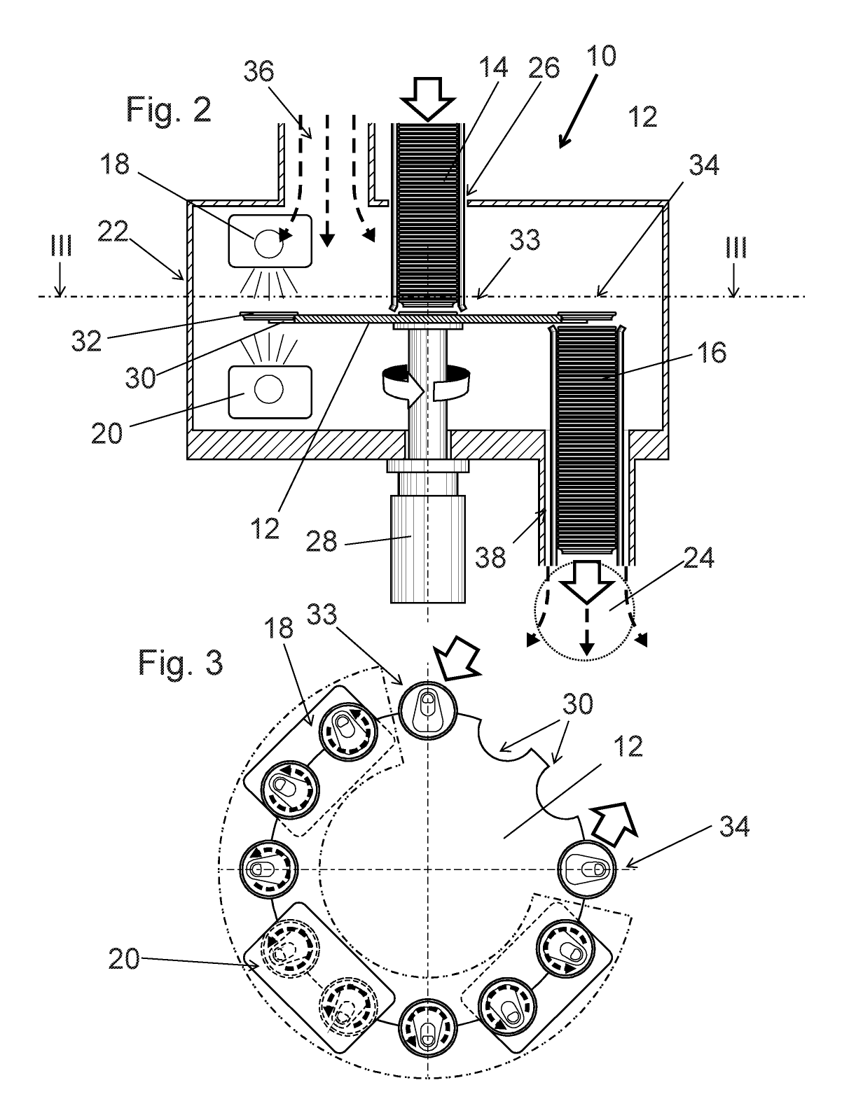

FIG. 2 shows a vertical section through the arrangement of FIG. 1, and

FIG. 3 shows a section III-III from FIG. 2.

According to FIGS. 1 to 3, the inventive arrangement 10 comprises a feed device 12 in the form of a transport star driven by a motor 28. The transport star 12 is arranged together with a first stacker device 14, a second stacker device 16, an upper UV emitter 18 and a lower UV emitter 20 in a substantially sealed housing 22. The first stacker device 14 is arranged in a feed opening 26 of the housing 22. The second stacker device 16 is arranged in an outlet pipe 38 which projects out of the housing 22. The outlet pipe 38 also forms a gas discharge for a process gas used in the housing 22, e.g. a sterile or sterilizing purging gas. A can closing unit 24 is arranged at the exit from the second stacker device 16.

Around its periphery, the transport star 12 has transport recesses 30 in which individual can lids 32 are accommodated. The direction of rotation of the transport star 12 is indicated by an arrow. The gas feed and the discharge 36, 38 can be provided optionally. The housing is preferably completely sealed except for the feed opening 26 for the can lids, the gas feed channel 36 and the outlet pipe 38 which forms both the can discharge opening and the gas discharge.

The inventive arrangement works as follows:

The can lids 32 are fed to the transport star 12 via the first stacker device 14 at the feed point 33. At the exit of the first stacker device 14 is arranged a descrambling device (not shown) which feeds the can lids 32 individually to the transport star, i.e. to the transport recesses of the transport star 12. The lids are then transported in the transport recesses 30 of the transport star 12, passing along beneath the upper UV emitter 18 and above the lower UV emitter 20 so that the can lids 32 are sterilized both on their upper and on their lower side. Finally, at the discharge point 34 the can lids are fed to the second stacker device 16 which forms the feed device for the closing unit 24 arranged beneath it. A gas, in particular a sterilizing gas, which is discharged at the outlet pipe 38 in which the second stacker device 16 is arranged, is also fed to the housing 22 through the gas feed channel 36. In this way, a sterile or sterilizing gas is washed over and around the can lids 32 during the entire descrambling, sterilization and feeding process so that the sterility produced by the UV emitters 18, 20 is maintained until the closing of a can with a sterilized can lid 32 in the can closing unit 24.

The dashed arrows in FIG. 2 indicate the path of the sterile gas which can be for example oxygen or CO₂ or any desired sterilizing gas.

The invention is not confined to the depicted exemplary embodiment but is capable of variation within the scope of the following claims.

### LIST OF REFERENCE CHARACTERS

10 Arrangement for disinfecting can lids and for closing cans

12 Feed device—transport star

14 First stacker device

16 Second stacker device

18 Upper UV emitter—first sterilization device

20 Lower UV emitter—second sterilization device

22 Housing

24 Can closing unit

26 Feed opening for can lids

28 Drive motor

30 Transport recess in the transport star for individual can lids

32 Can lids

33 Feed point of the conveyor path

34 Discharge point of the conveyor path

36 Gas feed channel

38 Outlet pipe

1-16. (canceled)

17. An arrangement for disinfecting can lids and for closing cans, the arrangement comprising:
a feed device formed with receiving portions for holding individual can lids;
said feed device being arranged in a feed region of a can closing unit; and
at least one sterilization device for the can lids that have been received individually in said feed device arranged in a conveying region of said feed device.

18. The arrangement according to claim 17, wherein said at least one sterilization device comprises a sterilizer disposed to sterilize a top and an underside of the individual can lids.

19. The arrangement according to claim 18, wherein said sterilization device comprises UV emitters for sterilizing the top and the underside of the individual can lids.

20. The arrangement according to claim 17, wherein said feed device comprises a transport star.

21. The arrangement according to claim 17, wherein the can closing unit is arranged in the conveying region of said feed device.

22. The arrangement according to claim 20, wherein said receiving portions are transport recesses for the individual can lids formed in a peripheral section of said transport star.

23. The arrangement according to claim 17, wherein said at least one sterilization device comprises at least one pulsed light device.

24. The arrangement according to claim 23, wherein a respective said pulsed light device is configured both above and below said feed device.

25. The arrangement according to claim 23, wherein said pulsed light device is a UV radiator.

26. The arrangement according to claim 17, further comprising a first stacker device for can lids arranged in an entry of the feed device.

27. The arrangement according to claim 26, further comprising a second stacker device disposed at an exit of said feed device and in the feed region of the can closing unit.

28. The arrangement according to claim 27, wherein said second stacker device forms an entry of the can closing unit.

29. The arrangement according to claim 17, wherein the can closing unit is disposed directly in or at an exit of said feed device.

30. The arrangement according to claim 17, wherein the can closing unit is disposed on said feed device and said feed device is configured to individually feed sterilized can lids to the can closing unit.

31. The arrangement according to claim 17, further comprising a sealed housing enclosing said feed device and said sterilization device, said sealed housing being formed with a gas feed and a gas discharge for a sterile gas, and with feed and discharge openings for the can lids.

32. The arrangement according to claim 31, wherein said feed opening for the can lids and said gas feed and/or said discharge opening for the can lids and said gas discharge are integrated.

33. The arrangement according to claim 17, further comprising a first stacker device for can lids arranged in an entry of the feed device, and a second stacker device disposed in said gas discharge at an entry to the can closing unit.
